(19) United States
(12) Patent Application Publication
(10) Pub. No.: US 2021/0348125 A1
(43) Pub. Date: Nov. 11, 2021

(54) INDUCED STEM MEMORY T CELLS AND METHODS OF USE THEREOF

(71) Applicant: Augusta University Research Institute, Inc., Augusta, GA (US)

(72) Inventors: Samir Khleif, Silver Spring, MD (US); Vivek Verma, Augusta, GA (US)

(21) Appl. No.: 17/379,778

(22) Filed: Jul. 19, 2021

Related U.S. Application Data
(62) Division of application No. 16/173,520, filed on Oct. 29, 2018.
(60) Provisional application No. 62/577,819, filed on Oct. 27, 2017.

(51) Int. Cl. C12N 5/0783; A61K 35/17; C07K 14/705; A61K 31/664; A61K 39/00; A61K 39/395; A61P 35/00; C07K 16/28

(52) U.S. Cl. CPC C12N 5/0636; A61K 2039/55516; C07K 14/70517; C07K 14/70585; C07K 14/70564; A61K 31/664; A61K 39/0011; A61K 39/3955; A61P 35/00; C07K 16/2878; A61K 39/39541; A61K 39/00118; C07K 2319/30; C12N 2501/999; A61K 2039/892; A61K 2039/876; A61K 2039/505; C07K 2317/75; A61K 35/17

(57) ABSTRACT

Methods for inducing CD8+ T cells to express a CD62L^{hi}CD44^{lo} naïve-like phenotype are provided. One embodiment provides a pharmaceutical composition containing CD8+ T cells induced to express a CD62L^{hi}CD44^{lo} naïve-like phenotype and optionally an excipient. The CD8+ T cells can be induced by contacting them with an effective amount of a MEK1/2 inhibitor. An exemplary MEK1/2 inhibitor is Selumetinib. The induced CD8+ cells can be used to treat cancer, reduce tumor burden, or treat infections in a subject in need thereof.

### CROSS REFERENCE TO RELATED APPLICATIONS

This application is a divisional application of U.S. patent application Ser. No. 16/173,520 filed on Oct. 29, 2018 and claims the benefit of and priority to U.S. Provisional Patent Application No. 62/577,819, filed on Oct. 27, 2017, and which is incorporated by reference in its entirety.

### REFERENCE TO SEQUENCE LISTING

The Sequence Listing submitted Oct. 29, 2018, as a text file named “064466.075_seqlisting_ST25.txt” created on Oct. 26, 2018, and having a size of 1.08 KB is hereby incorporated by reference pursuant to 37 C.F.R. § 1.52(e)(5).

### TECHNICAL FIELD OF THE INVENTION

Aspects of the invention are directed to compositions and methods for immunotherapy and in particular to methods of inducing activation of immune cells.

### BACKGROUND OF THE INVENTION

Targeted therapies using small molecule inhibitors are effective in treating various types of cancers (Jones, et al., Nat Rev Genetics, 17: 630-641 (2016)). Since the anti-tumor effects induced by the small molecule inhibitors are transient (Sierra, et al., Molecular Cancer, 9:7 5 (2010)), an understanding of the mechanisms of action of these inhibitors would better harness their anti-tumor potentials, alone or in combination with other therapies.

Over the recent years, the mechanistic link between T-cell function and metabolic programs has emerged as a therapeutic target in cancer patients (Pearce, et al., Science, 342: 1242454 (2103)). Interestingly, small molecule inhibitors and immune modulatory antibodies (Abs) have been found to affect the immune-cell physiology by reprogramming their metabolism (Patsoukis, N. et al., Nature Communications, 6, 6692 (2015); Jiang, Y., et al., Cell Death & Disease, 6: e1792 (2015); Sarkar, S. et al., J Exp Med, 205: 625-640 (2008); Overwijk, W. W., et al., J Exp Med, 188: 277-286 (1998)). To meet their energy demands, naïve T-cells use nutrients through mitochondrial oxidative phosphorylation (OXPHOS) while effector cells engage in aerobic glycolysis (Vander Heiden, M. G., et al., Science, 324: 1029-1033 (2009)). Once antigen is cleared, a small pool of memory cells is maintained through increased mitochondrial OXPHOS (Pearce, E. L., et al., Science, 342: 1242454 (2009)).

Memory T cells have an important role in the adaptive immune response to infectious diseases and cancer (Flynn, J. and P. Gorry, Clinical & Translational Immunology, 3, e20 (2014)). Some memory T cells demonstrate stem cell-like characteristics with their capacity to self-renew and also to generate more differentiated progeny from antigen stimulation. This T-cell subset, termed stem memory T cells (T_{SCM}) has been detected in CD4⁺ and CD8⁺ T-cell populations of mice, non-human primates (NHP) and humans. T_{SCM} display stem cell-like properties and constitute a small proportion of the memory T-cell subset, approximately 2-4% of the total CD4⁺ and CD8⁺ T-cell population in the blood. T_{SCM} have been described as representing the earliest and longest lasting developmental stage of memory T cells and exhibiting a gene profile which is between na\ddot{ï}ve and CM T cells (Flynn, J. and P. Gorry, Clinical & Translational Immunology, 3, e20 (2014)).

Thus it is an object of the invention to provide compositions and methods for inducing T_{SCM}.

It is another object to provide compositions and methods for improving adoptive cell transfer therapy.

### SUMMARY OF THE INVENTION

Methods and compositions for inducing CD8+ T cells to express a CD62L^{hi}CD44^{lo} naïve-like phenotype are provided. One embodiment provides a pharmaceutical composition containing CD8+ T cells induced to express a CD62L^{hi}CD44^{lo} naïve-like phenotype and optionally an excipient. The CD8+ T cells can be induced by contacting them with an effective amount of a MEK1/2 inhibitor. An exemplary MEK1/2 inhibitor is Selumetinib.

Another embodiment provides a method for inducing a stem cell memory T cells (T_{SCM}) like phenotype in CD8⁺ T-cells by contacting the CD8⁺ T-cells in vitro or ex vivo with an effective amount of an inhibitor of MEK1/2 to induce a T_{SCM} phenotype in the CD8⁺ T-cells; and optionally expanding the induced CD8⁺ T-cells in culture. The CD8+ T-cells express a CD62L^{hi}CD44^{lo} naïve-like CD8⁺ T-cells having elevated levels of Scal compared to untreated na\ddot{ï}ve cells. In one embodiment, the MEK1/2 inhibitor is Selumetinib.

The method of claim 10, wherein the T cell co-stimulatory receptor is selected from the group consisting of CD28, ICOS, HVEM, CD27, 4-1BB, OX40, DR3, GITR, CD30, CD2, 2B4, CD226, or a combination thereof.

One embodiment provides a method for reducing tumor burden in a subject in need thereof, by administering CD8+ cells induced to express a CD62L^{hi}CD44^{lo} naïve-like phenotype in the CD8⁺ T-cells in combination or alternation with an immunostimulatory agent, a potentiating agent, or a combination thereof in an amount effective to reduce the tumor burden in the subject. The immunostimulatory agent can be an antibody or antigen binding fragment thereof or a fusion protein that immunospecifically binds to and stimulates signal transduction through CD28, ICOS, HVEM, CD27, 4-1BB, OX40, DR3, GITR, CD30, CD2, 2B4, CD226, or a combination thereof. The potentiating agent can be cyclophosphamide.

One embodiment provides a method of adoptive cell transfer including contacting CD8⁺ T-cells ex vivo with an effective amount of a MEK1/2 inhibitor and an immunotherapeutic agent to induce a CD62L^{hi}CD44^{lo} naïve-like phenotype in the CD8⁺ T-cells, optionally expanding the induced CD8⁺ T-cells in culture; and administering the induced CD8⁺ T-cells to a subject in an amount effective to reduce tumor burden in the subject. The method optionally includes administering to the subject an immunostimulatory agent, a potentiating agent, or a combination thereof.

### BRIEF DESCRIPTION OF THE DRAWINGS

FIG. 1A shows a schedule of mice treatment. Mice bearing TC1 tumors were treated with MEKi for 15 days starting at day 6 (D6) post-tumor-implantation with intermittent tumor-specific vaccination on D12, 19 and 26. FIGS. 1B-1D show tumor volume and percent survival after various treatments as depicted for TC1 (FIGS. 1B-1C) and B16 (FIGS. 1D-1E). P-value in 1b is between MEKi and MEKi+ Vax. Data in (FIGS. 1B-1E) is representative of three experiments (n=8-10). FIG. 1F shows analysis of granzyme B⁺CD8⁺ cells and FIG. 1G shows analysis of E7⁺CD8⁺ T-cells in the TME of variously treated mice. A representative graph from one of three experiments is shown. FIGS. 1H-1K shows flow cytometric analysis of E7⁺ Granzyme⁺CD8⁺ cells and its statistical analysis (FIG. 1L). E7⁺ specific cells were stained by dextramer staining. Representative figure from one of the two experiments is shown. FIGS. 1M-1P show flow cytometry analysis of memory precursor (CD127⁺KLRG1⁻) and FIG. 1Q shows statistical analysis of (CD127⁺KLRG1⁻). FIG. 1R shows statistical analysis of short term memory (CD127⁻KLRG1⁺) cells in the TME in variously treated groups. Representative figure from one of two experiments is shown. *P<0.05, **P<0.01, ***P<0.001, ***P<0.0001

FIGS. 2A-2C show FACS analysis of in vitro activated pMel-CD8⁺ cells for memory characteristics after 48 h activation with g100₂₅₋₃₃ peptide alone (FIG. 2A) or in the presence of MEKi (FIG. 2b). FIGS. 2D-2F show representative micrographs and statistical analysis of proliferative ability of variously activated pMel-1 CD8⁺ T-cells, measured by dilution in VCT dye. FIGS. 2G-2H show expression of Sca1 on na\ddot{ï}ve T-cells (CD62L⁺ CD44⁻) after activation as in FIGS. 2A-2C. FIGS. 2I-2J show mitochondrial potential of CD62L⁺ CD44⁻CD8⁺ T-cells after respective activation as in FIGS. 2A-2C estimated by incorporation of TMRM dye. FIG. 2K shows mRNA expression levels of Klf2 and FIGS. 2L-2N show percentages of annexin⁺CD8⁺ cells in variously activated CD8⁺ cells. Experiments in FIGS. 2A to 2N were repeated at least three times in triplicate. Expression levels of memory markers is shown in FIGS. 2O-2Q as well as the mitochondrial potential in human CD8+ cells in FIGS. 2R-2U after 72 h of activation of total leukocytes with anti-CD3 antibody alone or in the presence of MEKi as indicated in the figure. Three experiments were repeated twice in triplicate. Representative micrographs from one of three experiments performed in triplicate are shown. (FIGS. 2R-2U and 2V-2X). Relative frequencies of CD62L⁺ CD44⁻ cells FIGS. 2V-2X and mitochondrial potential of CD8⁺ in which MEK1/2 was knocked down FIGS. 2Y-2AA). Data is representative of three experiments done at least in triplicate (mean±s.e.m.). *P<0.05, **P<0.01, ***P<0.001.

FIGS. 3A-3C show FACS micrographs showing incorporation of MitoFM dye during in vitro CD8⁺ T-cell activation using gp100₂₅₋₃₃ with (FIG. 3A) or without (FIG. 3B) MEKi treatment, and statistical analysis (FIG. 3C) of the numbers of CD8⁺ T-cells with high mitochondrial mass determined by MitoFM incorporation. FIG. 3D shows Mean fluorescence intensity (MFI) of NBDG expression in activated CD8⁺ T-cells as a measure of glucose uptake. FIG. 3E shows expression profiles of the Glutl gene, Slc2a1, in activated CD8⁺ T-cells. For results in FIGS. 3A-3E, representative figures from three experiments, done at least in triplicate, are shown. FIGS. 3F-3I show metabolic characteristics of MEKi-treated CD8⁺ T-cells showing OCR (FIGS. 3F, 3G), SRC (FIG. 3H) and ECAR (FIG. 3I) levels. Representative results from one of two experiments done in triplicate are shown. FIG. 3J shows expression levels of Cpt1a in MEKi-treated CD8⁺ cells. FIGS. 3K-3U show FACS micrographs and statistical analysis (FIG. 3U) of inhibition of proliferation after etomoxir treatment in MEKi-treated CD8⁺ T-cells. *P<0.05, **P<0.01, ***P<0.001.

FIGS. 4A-4C show FACS analysis of memory phenotype of activated cells (gp100 vs gp100 +MEKi) after antigen (Ag) re-challenge with gp100 peptide for 72 hrs. FIGS. 4D-4L shows estimation of expression of effector molecules in respectively re-challenged groups as described in FIG. 4A-4C. Results in FIGS. 4A-4L are representative of three replicated experiments done in triplicate. FIGS. 4M-4O show the scheme of ACT of B16 tumor-bearing mice with respectively activated CD8⁺ T-cells as indicated FIG. 4M, estimation of tumor volume FIG. 4N and percent survival FIG. 4O in treated groups. FIGS. 4P-4R show the estimation of numbers of adoptively transferred cells in spleens of treated mice 30 days after ACT as indicated. For the untreated (UT) and control (CTX) groups where the endpoint for euthanasia was reached earlier, the mice were sacrificed and spleens harvested and stored as single-cell suspensions at −80° C. At the end of the study, all samples were stained and processed together for FACS analysis. Representative of two experiments is shown. *P<0.05, **P<0.01, ***P<0.001.

FIG. 5A shows a treatment schedule of tumor bearing mice using MEKi and anti-OX40 Abs. Two tumor models namely TC1 and B16 were used. For TC1 tumors E7-peptide while for B16 tumors gp100₂₅₋₃₃ peptide was used as a vaccine. FIGS. 5B-5E show tumor growth profiles and respective mice survival graphs after various treatments in TC1 (FIGS. 5B-5C) and B16 (FIGS. 5D-5E) tumor models. Representative results from two experiments are shown. FIGS. 5F-5G show estimation of total (FIG. 5F) and antigen specific (FIG. 5G) CD8⁺ T-cells in the TC1-tumors of variously treated mice. Experiment was repeated twice and results from one representative experiment are shown. Each dot in FIGS. 5F-5G represents one mouse. FIG. 5H shows the treatment schedule for in vitro activation of pMel-1 CD8⁺ T-cells with various agents as shown in figure. FIGS. 5I-5X are flow cytometric plots showing the determination of CD8⁺ T-cell phenotype after re-stimulation of gp100₂₅₋₃₃+/− MEKi activated cells with gp100₂₅₋₃₃+/− anti-OX40-Abs as detailed in FIG. 5H. FIGS. 5I-5l shows the phenotype of CD8+ T-cells after respective treatments as shown in figure. FIGS. 5M-5X show expression profiles of the effector molecules and corresponding statistical analysis (FIGS. 5Y-5BB) after antigenic re-challenge as described in FIG. 5H. All experiments were repeated thrice in triplicated and representative data is shown. FIGS. 5CC-5FF show metabolic profiling of the gp100₂₅₋₃₃+/− MEKi activated cells after re-stimulation with gp100₂₅₋₃₃+/− anti-0X40-Abs. Experiment was repeated thrice in triplicate. *P<0.05, **P<0.01, ***P<0.001.

FIGS. 6A-6B show FACS micrographs showing the cell phenotype after 48 h activation in the presence of gp100 or gp100+MEKi. FIGS. 6B-6W show FACS analysis of expression of IFNγ, Granzyme, Perforin, KLRG1 and Eomes on CD62L⁺ CD44⁻ cells in FIGS. 6A-6B (marked by arrows). Data is representative of three experiments done in triplicates. FIGS. 6R-6V, show expression levels of mRNA of various effector and exhaustion markers after in vitro activation of pMel-1 CD8⁺ T-cells with gp100₂₅₋₃₃ with/without MEKi. mRNA was extracted from total CD8⁺ T-cell population after respective treatments. Experiment was repeated twice with similar results and representative results from one experiment are shown. *P<0.05, **P<0.01, ***P<0.001.

FIGS. 7A-7D shows confirmation of KD of MEK1 (FIG. 7A-7B) and MEK2 (FIG. 7C-7D) by FACS analysis after siRNA treatment. FIGS. 7E-7G show the memory phenotype generated after MEK1KD or MEK2KD in pMel-CD8⁺ T-cells. Data is representative of two experiments done in triplicates. *P<0.05, **P<0.01.

FIGS. 8A-8C show in vitro estimation of mitochondrial reactive oxygen species in MEKi-treated CD8⁺ T-cells activated with gp100₂₅₋₃₃ peptide for 48 h. FIG. 8D-8N shows the determination of effect of inhibition of mitochondrial respiration by oligomycin on proliferation of gp100₂₅₋₃₃ activated MEKi-treated CD8⁺ T-cells. Representative figure from two experiments done in triplicate are shown. Statistics were calculated by Student's t test for unpaired means. **P<0.01, ***P<0.001.

FIGS. 9A-9H show FACS analysis of expression patterns of effector molecules after incubation of activated cells (gp100 Vs gp100+MEKi) with IL2 (FIGS. 9A-9D) or anti-OX40 Ab (FIGS. 9E-9H) for 72 hrs.

### DETAILED DESCRIPTION OF THE INVENTION

### I. Definitions

The term “immunostimulatory agent” refers to a substance that stimulates or activates an immune response. Stimulating or activating an immune response includes inhibiting a suppressive immune response.

The term “immunosuppressive agent” refers to a substance that suppresses or inhibits an immune response.

The “term co-stimulatory agent” refers to a substance that binds to a receptor on a T cell that results in an immune stimulatory response. A co-stimulatory agent does not induce or activate a suppressive immune response. Stimulating or activating an immune response includes inhibiting a suppressive immune response.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, a molecule is said to be able to “immunospecifically bind” a second molecule if such binding exhibits the specificity and affinity of an antibody to its cognate antigen. Antibodies are said to be capable of immunospecifically binding to a target region or conformation (“epitope”) of an antigen if such binding involves the antigen recognition site of the immunoglobulin molecule. An antibody that immunospecifically binds to a particular antigen may bind to other antigens with lower affinity if the other antigen has some sequence or conformational similarity that is recognized by the antigen recognition site as determined by, e.g., immunoassays, BIACORE® assays, or other assays known in the art, but would not bind to a totally unrelated antigen. In some embodiments, however, antibodies (and their antigen binding fragments) will not cross-react with other antigens. Antibodies may also bind to other molecules in a way that is not immunospecific, such as to FcR receptors, by virtue of binding domains in other regions/domains of the molecule that do not involve the antigen recognition site, such as the Fc region.

As used herein, a molecule is said to “physiospecifically bind” a second molecule if such binding exhibits the specificity and affinity of a receptor to its cognate binding ligand. A molecule can be capable of physiospecifically binding to more than one other molecule.

As used herein, the term “antibody” is intended to denote an immunoglobulin molecule that possesses a “variable region” antigen recognition site. The term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196: 901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26: 230; Nuttall et al., 2000, Cur. Pharm. Biotech. 1: 253; Reichmann and Muyldermans, 1999, J. Immunol. Meth. 231: 25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-ID) antibodies (including, e.g., anti-Id and anti-anti-Id antibodies to antibodies). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab′, F(ab′)₂, Fv, single chain (ScFv), and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.).

As used herein the term “modulate” relates to a capacity to alter an effect, result, or activity (e.g., signal transduction). Such modulation can be agonistic or antagonistic. Antagonistic modulation can be partial (i.e., attenuating, but not abolishing) or it can completely abolish such activity (e.g., neutralizing). Modulation can include internalization of a receptor following binding of an antibody or a reduction in expression of a receptor on the target cell. Agonistic modulation can enhance or otherwise increase or enhance an activity (e.g., signal transduction). In a still further embodiment, such modulation can alter the nature of the interaction between a ligand and its cognate receptor so as to alter the nature of the elicited signal transduction. For example, the molecules can, by binding to the ligand or receptor, alter the ability of such molecules to bind to other ligands or receptors and thereby alter their overall activity. In some embodiments, such modulation will provide at least a 10% change in a measurable immune system activity, at least a 50% change in such activity, or at least a 2-fold, 5-fold, 10-fold, or at least a 100-fold change in such activity.

The term “substantially,” as used in the context of binding or exhibited effect, is intended to denote that the observed effect is physiologically or therapeutically relevant. Thus, for example, a molecule is able to substantially block an activity of a ligand or receptor if the extent of blockage is physiologically or therapeutically relevant (for example if such extent is greater than 60% complete, greater than 70% complete, greater than 75% complete, greater than 80% complete, greater than 85% complete, greater than 90% complete, greater than 95% complete, or greater than 97% complete). Similarly, a molecule is said to have substantially the same immunospecificity and/or characteristic as another molecule, if such immunospecificities and characteristics are greater than 60% identical, greater than 70% identical, greater than 75% identical, greater than 80% identical, greater than 85% identical, greater than 90% identical, greater than 95% identical, or greater than 97% identical).

As used herein, the term “cancer” refers to a neoplasm or tumor resulting from abnormal uncontrolled growth of cells. The term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or web-like matrices in a three-dimensional basement membrane or extracellular matrix preparation. Non-cancer cells do not form colonies in soft agar and form distinct sphere-like structures in three-dimensional basement membrane or extracellular matrix preparations.

As used herein, an “immune cell” refers to any cell from the hemopoietic origin including, but not limited to, T cells, B cells, monocytes, dendritic cells, and macrophages.

As used herein, the terms “immunologic,” “immunological” or “immune” response is the development of a beneficial humoral (antibody mediated) and/or a cellular (mediated by antigen-specific T cells or their secretion products) response directed against a peptide in a recipient patient. Such a response can be an active response induced by administration of immunogen or a passive response induced by administration of antibody or primed T-cells. A cellular immune response is elicited by the presentation of polypeptide epitopes in association with Class I or Class II WIC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

As used herein, the terms “individual,” “host,” “subject,” and “patient” are used interchangeably herein, and refer to a mammal, including, but not limited to, humans, rodents, such as mice and rats, and other laboratory animals.

The “term co-inhibitory agent” refers to a substance that binds to a receptor on a T cell that results in an immune suppressive response. A co-inhibitory agent does not induce or activate an activating or stimulatory immune response.

### II. Compositions for Immunotherapy

One embodiment provides a pharmaceutical composition containing T cells induced to have a CD62L^{hi}CD44^{lo} naïve-like phenotype. The composition can be administered to a subject in need thereof to enhance or promote a stimulatory or activating immune response. One embodiment provides T cells that are induced to have a T_{SCM} like phenotype.

### A. T_{SCM}

Recently, a distinct subset of memory cells termed stem-cell memory (T_{SCM}) cells has been described. Phylogenetically, T_{SCM} cells are placed between na\ddot{ï}ve and memory cells (Fuertes Marraco, S. A., et al., Sci Transl Med, 7: 282ra248 (2015)). However, T_{SCM} cells can be distinguished from memory cells by their decreased mitochondrial membrane potential and lower expression of CD44 (Sukumar, M., et al., Cell Metabolism, 23: 63-76 (2016)), while they can be differentiated from na\ddot{ï}ve T-cells by their high expression of activation markers such as CD25 and Scal (Rosenblum, M. D., et al., Nat Rev Immunol, 16: 90-101 (2016)). Functionally, T-cells having the T_{SCM} phenotype have been shown to have enhanced anti-tumor responses compared to both na\ddot{ï}ve and memory T-cells (Golubovskaya, V., and Wu, L., Cancers, 8(3): 36 (2016)), which seem to depend upon their long-term persistence, self-renewability and ability to differentiate into effector T-cells (T_{EFF}) (Graef, P., et al., Immunity, 41: 116-126 (2014)).

### B. MEK1/2 Inhibitors

T cells can be induced to have a T_{SCM} like phenotype by contacting the T cells with a MEK1/2 inhibitor. The MEK1/2 inhibitor can be added to T cells ex vivo or administered to a subject in need thereof. Typically the MEK1/2 inhibitor is added to a T cell culture and the induced T cells culture until the Tcells develop a CD62LhiCD44lo naïve-like phenotype.

In one embodiment, the MEK1/2 inhibitor is TAK-733. TAK-733 is a potent and selective MEK allosteric site inhibitor for MEK1 with IC₅₀ of 3.2 nM, inactive to Abl1, AKT3, c-RAF, CamK1, CDK2, c-Met, etc.

In one embodiment, the MEK1/2 inhibitor is Selumetinib. Selumetinib (AZD6244) is a potent, highly selective MEK1 inhibitor with IC₅₀ of 14 nM, also inhibits ERK1/2 phosphorylation with IC₅₀ of 10 nM, no inhibition to p38a, MKK6, EGFR, ErbB2, ERK2, B-Raf, etc.

In one embodiment, the MEK1/2 inhibitor is PD98059. PD98059 is a non-ATP competitive MEK inhibitor with IC₅₀ of 2 μM, specifically inhibits MEK-1-mediated activation of MAPK; does not directly inhibit ERK1 or ERK2.

In one embodiment, the MEK1/2 inhibitor is Trametinib. Trametinib (GSK1120212) is a highly specific and potent MEK1/2 inhibitor with IC₅₀ of 0.92 nM/1.8 nM, no inhibition of the kinase activities of c-Raf, B-Raf, or ERK1/2.

In one embodiment, the MEK1/2 inhibitor is PD184352. PD184352 (CI-1040) is an ATP non-competitive MEK1/2 inhibitor with IC₅₀ of 17 nM, 100-fold more selective for MEK1/2 than MEK5.

In one embodiment, the MEK1/2 inhibitor is Refametinib. Refametinib (RDEA119, Bay 86-9766) is a potent, ATP non-competitive and highly selective inhibitor of MEK1 and MEK2 with IC50 of 19 nM and 47 nM, respectively.

In one embodiment, the MEK1/2 inhibitor is U0126-EtOH. U0126-EtOH is a highly selective inhibitor of MEK1/2 with IC₅₀ of 0.07 μM/0.06 μM, 100-fold higher affinity for ΔN3-S218E/S222D MEK than PD98059.

In one embodiment, the MEK1/2 inhibitor is SL327. SL327 is a selective inhibitor for MEK1/2 with IC₅₀ of 0.18 μM/ 0.22 μM, no activity towards Erk1, MKK3, MKK4, c-JUN, PKC, PKA, or CamKII; capable of transport through the blood-brain barrier.

### C. Immune Stimulatory Agents

The induced T cell compositions can be administered to a subject in combination or alternation with one or more immune stimulatory agents. Representative immune stimulatory agents include, but are not limited to antibodies or fusion proteins that activate CD27, CD40, OX40, GITR, CD137, CD28, or ICOS signal transduction.

### D. Co-Therapies

The induced T cell compositions can be administered to a subject in combination or alternation with one or more co-therapies.

### 1. Potentiating Agents

In some embodiments, the induced T cell compositions can be administered to a subject in combination or alternation with a potentiating agent. The potentiating agent acts to increase efficacy the immune response up-regulator, possibly by more than one mechanism, although the precise mechanism of action is not essential to the broad practice of the present invention.

In some embodiments, the potentiating agent is cyclophosphamide. Cyclophosphamide (CTX, Cytoxan®, or Neosar®) is an oxazahosphorine drug and analogs include ifosfamide (IFO, Ifex), perfosfamide, trophosphamide (trofosfamide; Ixoten), and pharmaceutically acceptable salts, solvates, prodrugs and metabolites thereof (US patent application 20070202077 which is incorporated in its entirety). Ifosfamide (MITOXANA®) is a structural analog of cyclophosphamide and its mechanism of action is considered to be identical or substantially similar to that of cyclophosphamide. Perfosfamide (4-hydroperoxycyclophosphamide) and trophosphamide are also alkylating agents, which are structurally related to cyclophosphamide. For example, perfosfamide alkylates DNA, thereby inhibiting DNA replication and RNA and protein synthesis. New oxazaphosphorines derivatives have been designed and evaluated with an attempt to improve the selectivity and response with reduced host toxicity (Liang J, et al., Curr Pharm Des. 13(9): 963-78 (2007)). These include mafosfamide (NSC 345842), glufosfamide (D19575, beta-D-glucosylisophosphoramide mustard), S-(-)-bromofosfamide (CBM-11), NSC 612567 (aldophosphamide perhydrothiazine) and NSC 613060 (aldophosphamide thiazolidine). Mafosfamide is an oxazaphosphorine analog that is a chemically stable 4-thioethane sulfonic acid salt of 4-hydroxy-CPA. Glufosfamide is IFO derivative in which the isophosphoramide mustard, the alkylating metabolite of IFO, is glycosidically linked to a beta-D-glucose molecule. Additional cyclophosphamide analogs are described in U.S. Pat. No. 5,190,929 entitled “Cyclophosphamide analogs useful as anti-tumor agents” which is incorporated herein by reference in its entirety.

While CTX itself is nontoxic, some of its metabolites are cytotoxic alkylating agents that induce DNA crosslinking and, at higher doses, strand breaks. Many cells are resistant to CTX because they express high levels of the detoxifying enzyme aldehyde dehydrogenase (ALDH). CTX targets proliferating lymphocytes, as lymphocytes (but not hematopoietic stem cells) express only low levels of ALDH, and cycling cells are most sensitive to DNA alkylation agents.

Low doses of CTX (<200 mg/kg) can have immune stimulatory effects, including stimulation of anti-tumor immune responses in humans and mouse models of cancer (Brode & Cooke Crit Rev. Immunol. 28: 109-126 (2008)). These low doses are sub-therapeutic and do not have a direct anti-tumor activity. In contrast, high doses of CTX inhibit the anti-tumor response. Several mechanisms may explain the role of CTX in potentiation of anti-tumor immune response: (a) depletion of CD4+CD25+FoxP3+ Treg (and specifically proliferating Treg, which may be especially suppressive), (b) depletion of B lymphocytes; (c) induction of nitric oxide (NO), resulting in suppression of tumor cell growth; (d) mobilization and expansion of CD11b+Gr−1+ MDSC. These primary effects have numerous secondary effects; for example following Treg depletion macrophages produce more IFN-γ and less IL-10. CTX has also been shown to induce type I IFN expression and promote homeostatic proliferation of lymphocytes.

Treg depletion is most often cited as the mechanism by which CTX potentiates the anti-tumor immune response. This conclusion is based in part by the results of adoptive transfer experiments. In the AB1-HA tumor model, CTX treatment at Day 9 gives a 75% cure rate. Transfer of purified Treg at Day 12 almost completely inhibited the CTX response (van der Most et al. Cancer Immunol. Immunother. 58 :1219-1228 (2009). A similar result was observed in the HHD2 tumor model: adoptive transfer of CD4+CD25+ Treg after CTX pretreatment eliminated therapeutic response to vaccine (Taieb, J. J. Immunol. 176: 2722-2729 (2006)).

Numerous human clinical trials have demonstrated that low dose CTX is a safe, well-tolerated, and effective agent for promoting anti-tumor immune responses (Bas, & Mastrangelo Cancer Immunol. Immunother. 47: 1-12 (1998)).

The optimal dose for CTX to potentiate an anti-tumor immune response, is one that lowers overall T cell counts by lowering Treg levels below the normal range but is subtherapeutic (see Machiels et al. Cancer Res. 61: 3689-3697 (2001)).

In human clinical trials where CTX has been used as an immunopotentiating agent, a dose of 300 mg/m² has usually been used. For an average male (6 ft, 170 pound (78 kg) with a body surface area of 1.98 m²), 300 mg/m²is 8 mg/kg, or 624 mg of total protein. In mouse models of cancer, efficacy has been seen at doses ranging from 15-150 mg/kg, which relates to 0.45-4.5 mg of total protein in a 30 g mouse (Machiels et al. Cancer Res. 61: 3689-3697 (2001), Hengst et al Cancer Res. 41: 2163-2167 (1981), Hengst Cancer Res. 40: 2135-2141 (1980)).

For larger mammals, such as a primate, such as a human, patient, such mg/m² doses may be used but unit doses administered over a finite time interval may also be used. Such unit doses may be administered on a daily basis for a finite time period, such as up to 3 days, or up to 5 days, or up to 7 days, or up to 10 days, or up to 15 days or up to 20 days or up to 25 days, are all specifically contemplated by the invention. The same regimen may be applied for the other potentiating agents recited herein.

In other embodiments, the potentiating agent is an agent that reduces activity and/or number of regulatory T lymphocytes (T-regs), such as Sunitinib (SUTENT®), anti-TGFβ or Imatinib (GLEEVAC®). The recited treatment regimen may also include administering an adjuvant.

Useful potentiating agents also include mitosis inhibitors, such as paclitaxol, aromatase inhibitors (e.g. Letrozole) and angiogenesis inhibitors (VEGF inhibitors e.g. Avastin, VEGF-Trap) (see, for example, Li et al., Clin Cancer Res. 12(22): 6808-16 (2006), anthracyclines, oxaliplatin, doxorubicin, TLR4 antagonists, and IL-18 antagonists.

2. Chemotherapeutic Agents

The induced T cell compositions can be administered to a subject in combination or alternation with one or more chemotherapeutic agents and/or pro-apoptotic agents. Representative chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2) and combinations thereof.

### D. Pharmaceutical Formulations

The induced T cells can be formulated as a pharmaceutical composition for parenteral administration. The induced T cells are typically administered in an aqueous solution, by parenteral injection. The formulation may also be in the form of a suspension or emulsion. In general, pharmaceutical compositions are provided including effective amounts induced T cells, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., ascorbic acid, sodium metabisulfite), and preservatives (e.g., Thimersol, benzyl alcohol) and bulking substances (e.g., lactose, mannitol).

### E. Vaccines

The disclosed induced T cells can be administered as part of a vaccine composition. In one embodiment, the vaccine contains a tumor specific antigen. The antigen expressed by the tumor may be specific to the tumor, or may be expressed at a higher level on the tumor cells as compared to non-tumor cells. Antigenic markers such as serologically defined markers known as tumor associated 30 antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.) (see, e.g., U.S. Pat. No. 6,699,475; Jager, et al., Int. J. Cancer, 106: 817-20 (2003); Kelmedy, et al., Int. Rev. Immunol., 22: 141-72 (2003); Scanlan, et al. Cancer Immun., 4: 1 (2004)).

Genes that encode cellular tumor associated antigens include cellular oncogenes and proto-oncogenes that are aberrantly expressed. In general, cellular oncogenes encode products that are directly relevant to the transformation of the cell, and because of this, these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309: 883 (1983) Lloyd, et al., Int. J. Cane., 71: 842 (1997)). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (S1N), and placental alkaline phosphatase (PLAP), in efforts to provide diagnostic and/or prognostic profiles of ovarian and other carcinomas (e.g., Sarandakou, et al., Acta Oncol., 36: 755 (1997)˜Sarandakou, et aL, Eur. J. Gynecol. Oncol, 19: 73 (1998); Meier, et al., Anticancer Res., 17(48): 2945 (1997); Kudoh, et al., Gynecol. Obstet. Invest., 47 :52 (1999)). Elevated serum CA125 may also accompany neuroblastoma (e.g., Hirokawa, et al., Surg. Today, 28: 349 (1998), while elevated CEA and SCC, among others, may accompany colorectal cancer (Gebauer, et al., Anticancer Res., 17(48): 2939 (1997)).

The tumor associated antigen, mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52: 181 (1992); Chang, et al., Int. J. Cancer, 50: 373 (1992); Chang, et al., Int J Cancer, 51: 548 (1992); Chang, et al., Proc. Natl. Acad. Sci. USA, 93: 136 (1996); Chowdhury, et al., Proc. Natl. Acad. Sci. USA, 95: 669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50: 373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may include a cell surface molecule. Tumor antigens of known structure and having a known or described function, include the following cell surface receptors: HER1 (GenBank Accession No. U48722), HER2 (Yoshino, et al., J Immunol., 152: 2393 (1994).

Additional tumor associated antigens include prostate surface antigen (PSA) (U.S. Pat. Nos. 6,677,157; 6,673,545); -human chorionic gonadotropin -HCG) (McManus, et al., Cancer Res., 36: 3476-81 (1976); Yoshimura, et al., Cancer, 73: 2745-52 (1994); Yamaguchi, et al., Br. J Cancer, 60: 382-84 (1989): Alfthan, et al., Cancer Res., 52: 4628-33 (1992)); glycosyltransferase-1,4-N-acetylgalactosaminyltransferases (GalNAc) (Hoon, et al., Int. J Cancer, 43: 857-62 (1989); Ando, et al., Int. J Cancer, 40: 12-17 (1987); Tsuchida, et aL, J Nat. Cancer, 78: 45-54 (1987); Tsuchida, et al., J Natl. Cancer, 78: 55-60 (1987)); NUC18 (Lehmann, et al., Proc. Natl. Acad. Sci. USA, 86: 9891-95 (1989); Lehmann, et al., Cancer Res., 47: 841-45 (1987)); melanoma antigen gp75 (Vijayasardahi, et al., J Exp. Med., 171: 1375-80 (1990); GenBankAccession No. X51455); human cytokeratin 8; high molecular weight melanoma antigen (Natali, et al., Cancer, 59: 55-63 (1987); keratin 19 (Datta, et al., J. Clin. Oncol., 12: 475-82 (1994)).

Tumor antigens of interest include antigens regarded in the art as “cancer/testis” (CT) antigens that are immunogenic in subjects having a malignant condition (Scanlan, et aL, Cancer Immun., 4: 1 (2004)). CT antigens include at least 19 different families of antigens that contain one or more members and that are capable of inducing an immune response, including but not limited to MAGEA (CT1); BAGE (CT2); MAGEB (CT3); GAGE (CT4); SSX (CT5); NY-ES0-1 (CT6); MAGEC (CT7); SYCP1 (C8); SPANXB 1 (CT11.2); NA88 (CT18); CTAGE (CT21); SP A17 (CT22); OYTES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor associated or tumor-specific antigen, include, but not limited to, alphaactinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferase AS fusion protein, HLA-A2, HLA-All, hsp70-2, KIAA0205, Mart2, Mum-1, 2, and 3, neo-PAP, myosin class I, OS-9, pm1RARa fusion protein, PTPRK, K-ras, N-ras, Triosephosphate isomerase, Bage-1, Gage 3,4,5,6,7, GnTV, Herv-K-mel, Lage-1, Mage-S A1,2,3,4,6,10,12, Mage-C2, NA-88, NY-Eso-1/Lage-2, SP17, SSX-2, and TRP2-Int2, MelanA (MART-I), gp100 (Pme117), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Me1-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, -Catenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS.

### III. Methods of Use

### A. Inducing T_{SCM} Like Phenotypes in T Cells

One embodiment provides a method for inducing a stem cell memory T cell (T_{SCM}) phenotype in CD8+ T-cells by contacting the CD8+ T-cells in vitro or ex vivo with an effective amount of an inhibitor of MEK1/2 to induce a T_{SCM} phenotype in the CD8+ T-cells. The induced T cells can be expanded in cell culture. In one embodiment, the induced T cells have a CD62L^{hi}CD44^{lo} naïve-like phenotype, elevated levels of Scal compared to untreated na\ddot{ï}ve cells, or both. The MEK1/2 inhibitor can be one or more of the inhibitors described above. In one embodiment, the MEK1/2 inhibitor is Selumetinib.

The induced T cells can be harvested from cell culture and aliquoted into suitable containers for sale or distribution. Alternatively, the induced T cells can be cryopreserved using conventional techniques.

In one embodiment the induced T cells are genetically engineered to bind to a target protein or peptide. The target protein or peptide can be a tumor specific antigen or a viral specific antigen. Exemplary tumor specific antigens are described above. In one embodiment the induced T cells express a chimeric antigen receptor.

### B. Methods of Treatment

The induced T cells can be administered to a subject in need thereof, for example as part of a treatment for cancer, a tumor, or an infection. In one embodiment, the induced T cells are autologous T cells. The autologous T cells can be genetically engineered to target tumor cells prior to administration to the subject.

In another embodiment, the induced T cells are administered in combination or alternation with a second therapeutic agent. Exemplary second therapeutic agents include, but are not limited to immunostimulatory agents, chemotherapeutic agents, adjuvants, vaccines, tumor antigen, viral antigens, potentiating agents, or combinations thereof. The immunostimulatory agent can be an antibody, or antigen binding fragment thereof or a fusion protein that immunospecifically binds to and induces signal transduction through CD28, ICOS, HVEM, CD27, 4-1BB, OX40, DR3, GITR, CD30, CD2, 2B4, CD226, or a combination thereof.

In one embodiment, the induced T cells are administered in combination or alternation with a potentiating agent such as cyclophosphamide.

Another embodiment provides a method for adoptive cell transfer therapy that includes harvesting CD8+ T cells from a subject, contacting the harvested CD8+ T cells with an effective amount of a MEK1/2 inhibitor to induce a CD62L^{hi}CD44^{lo} naïve-like phenotype, and administering the induced T cells to the subject. The induced T cells can be optionally expanded in culture prior to administration. In one embodiment, the MEK1/2 inhibitor is Selumetinib.

The T cells to be induced can be obtained by culturing a tumor biopsy from the subject in the presence of IL-2 to stimulate the growth of T cells that specifically target and kill the tumor cells. The tumor specific T cells can be harvested from culture and purified if necessary. The harvested T cells can be expanded in cell culture prior to administration to the subject. Additionally, the tumor specific T cells can be genetically modified to express a chimeric antigen receptor or a binding moiety to a target protein. Another embodiment provides a method for reducing tumor burden in a subject in need thereof by contacting CD8+ T-cells ex vivo with an effective amount of a MEK1/2 inhibitor to induce a CD62L^{hi}CD44^{lo} naïve-like phenotype in the CD8+ T-cells, optionally expanding the induced CD8+ T-cells in culture, and administering the induced CD8+ T-cells to the subject in an amount effective to reduce tumor burden in the subject. The induced T cells can be administered in combination or alternation with an immunostimulatory agent, a potentiating agent or both.

### 1. Treatment of Cancer

Specific cancers and related disorders that can be treated or prevented by methods and compositions disclosed herein include, but are not limited to, leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as, but not limited to, Hodgkin's disease or non-Hodgkin's disease lymphomas (e.g., diffuse anaplastic lymphoma kinase (ALK) negative, large B-cell lymphoma (DLBCL); diffuse anaplastic lymphoma kinase (ALK) positive, large B-cell lymphoma (DLBCL); anaplastic lymphoma kinase (ALK) positive, ALK+ anaplastic large-cell lymphoma (ALCL), acute myeloid lymphoma (AML)); multiple myelomas such as, but not limited to, smoldering multiple myeloma, non-secretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, carcinosarcoma. In addition, cancers include myxosarcoma, osteogenic sarcoma, endotheliosarcoma, lymphangio endothelio sarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

### 2. Treatment of Infections

The disclosed compositions and methods can be used to treat infections and infectious diseases. The infection or disease can be caused by a bacterium, virus, protozoan, helminth, or other microbial pathogen that enters intracellularly and is attacked, i.e., by cytotoxic T lymphocytes.

The infection or disease can be acute or chronic. An acute infection is typically an infection of short duration. During an acute microbial infection, immune cells begin expressing immunomodulatory receptors. Accordingly, in some embodiments, the method includes increasing an immune stimulatory response against an acute infection.

The infection can be caused by, for example, but not limited to Candida albicans, Listeria monocytogenes, Streptococcus pyogenes, Streptococcus pneumoniae, Neisseria meningitidis, Staphylococcus aureus, Escherichia coli, Acinetobacter baumannii, Pseudomonas aeruginosa or Mycobacterium.

In some embodiments, the disclosed compositions are used to treat chronic infections, for example infections in which T cell exhaustion or T cell anergy has occurred causing the infection to remain with the host over a prolonged period of time. Exemplary infections to be treated are chronic infections caused by a hepatitis virus, a human immunodeficiency virus (HIV), a human T-lymphotrophic virus (HTLV), a herpes virus, an Epstein-Barr virus, or a human papilloma virus.

Because viral infections are cleared primarily by T cells, an increase in T-cell activity would be therapeutically useful in situations where more rapid or thorough clearance of an infective viral agent would be beneficial to an animal or human subject. Thus, the disclosed compositions can be administered for the treatment of local or systemic viral infections, including, but not limited to, immunodeficiency (e.g., HIV), papilloma (e.g., HPV), herpes (e.g., HSV), encephalitis, influenza (e.g., human influenza virus A), and common cold (e.g., human rhinovirus) and other viral infections, caused by, for example, HTLV, hepatitis virus, respiratory syncytial virus, vaccinia virus, and rabies virus. The molecules can be administered topically to treat viral skin diseases such as herpes lesions or shingles, or genital warts. The molecules can also be administered systemically to treat systemic viral diseases, including, but not limited to, AIDS, influenza, the common cold, or encephalitis.

Representative infections that can be treated, include but are not limited to infections cause by microorganisms including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni.

Other microorganisms that can be treated using the disclosed compositions and methods include, bacteria, such as those of Klebsiella, Serratia, Pasteurella; pathogens associated with cholera, tetanus, botulism, anthrax, plague, and Lyme disease; or fungal or parasitic pathogens, such as Candida (albicans, krusei, glabrata, tropicalis, etc.), Cryptococcus, Aspergillus (fumigatus, niger, etc.), Genus Mucorales (mucor, absidia, rhizophus), Sporothrix (schenkii), Blastomyces (dermatitidis), Paracoccidioides (brasiliensis), Coccidioides (immitis) and Histoplasma (capsulatuma), Entamoeba, histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Toxoplasma gondi, etc.), Sporothrix, Blastomyces, Paracoccidioides, Coccidioides, Histoplasma, Entamoeba, Histolytica, Balantidium, Naegleria, Acanthamoeba, Giardia, Cryptosporidium, Pneumocystis, Plasmodium, Babesia, or Trypanosoma, etc.

### IV. Kits

One embodiment provides a kit. The kit contains one or more MEK1/2 inhibitors and induced T cells having a CD62L^{hi}CD44^{lo} naïve-like phenotype in one or more containers. The kit may include instructions or labels promoting or describing the use of the compounds of the invention.

### EXAMPLES

### Example 1: MEKi Enhances Expansion of Antigen-Specific CD8⁺ T-Cells and Enriches Memory Precursors in the Tumor Microenvironment Leading to Reduced Tumor Growth

### Methods and Materials

Mice and cell culture. 4-6-week-old C57BL/6 mice (wild-type (WT) and pMel-1) from Jackson Laboratory or in-house bred pMel-1 mice with transgenic CD8⁺ T-cells having TCR from melanoma-specific gp100₂₅₋₃₃ peptide were used as outlined in various experiments. Animals had free access to water and food. All experiments were performed under protocols approved by the Augusta University Georgia Cancer Center Institutional Animal Care and Use Committee (IACUC). Cancer cell lines used in the present study included TC1 (kindly provided by Dr. T-C Wu at Johns Hopkins University) and B16-melanoma (obtained from American Type Culture Collection (ATCC)). Cell lines were routinely tested for absence of any contamination, including mycoplasma, by microscopic evaluation and PCR-based methods. Primary murine CD8⁺ cells were isolated by fluorescence-activated cell sorting (FACS) and in some cases by negative selection using magnetic beads (Miltenyi Biotec) and cultured in RPMI 1640 medium supplemented with 10% FBS, 2 mM glutamine, 10 mM HEPES and 55 μM β-mercaptoethanol. All cell populations were greater than 95% pure.

Tumor establishment and mice treatment. Mice were injected with 70,000 TC1 cells/mouse or 2×10⁵ B16 cells/mouse in the right flank. Treatment in respective groups was started when tumors reached an average size of approximately 0.075 cm³. In a few experiments, mouse treatment was started when tumors reached an average size of 0.125-0.150 cm³. MEKi treatment was done using selumetinib obtained from AstraZeneca. Mice were dosed orally for fifteen days starting at day 6-7 (at an average tumor size of 0.04-0.06 cm³) at a dose of 10 mg/Kg. For vaccination, TC1-specific E7-peptide (RAHYNIVTF (SEQ ID NO:1); 100 μg/mouse/100 μl) or B16-specific gp100₂₅₋₃₃ peptide (KVPRNQDWL (SEQ ID NO:2); 100 μg/mouse/100 μl) was mixed with a pan HLA DR-binding epitope (PADRE; aK-Cha-VAAWTLKAAa (SEQ ID NO:3), 20 μg/mouse) and QuilA (10 μg/mouse). Mice were vaccinated twice with a one-week interval starting at day 12-13. In some groups, mice were treated with 200 μg/mouse of anti-OX40-Ab (clone OX86) every third day starting with the first vaccine.

PCR analysis. Total RNA was extracted from gp100₂₅₋₃₃ or gp100₂₅₋₃₃+ MEKi activated CD8⁺ T-cells using TRIzol reagent (Invitrogen), and dissolved in RNase-free water. 1 μg total RNA was subjected to single-strand cDNA synthesis using iScript™ cDNA Synthesis Kit (BioRad Inc., USA). Data were procured using StepOnePlus Real-Time PCR System from Applied Biosystems and normalized to the geometric mean of the housekeeping gene beta-actin.

Tumor harvest and sample preparation. Two-to-three days after the second vaccination, mice in the various groups were sacrificed, and tumors were harvested. Chopped tumors were suspended in enzymatic solution of liberase (5 mg/ml) and DNase I (100 μg/ml) followed by incubation at 37° C./30 min with intermittent shaking. Samples were mashed through a 70 μm cell strainer and finally suspended in FACS buffer (PBS+2.5% FBS) and processed for FACS staining.

Flow cytometry analyses. Flow cytometry was done on a BD LSR II Flow Cytometer. Antibodies used included anti-CD8, anti-CD25, anti-Sca1, anti-granzyme, anti-perforin, anti-KLRG1, anti-CD62L, anti-CD44, anti-CD127, anti-CCR7, anti-CD45RO, and anti-IFNγ. VioleT-cell Trace (VCT) and fixable Live/Dead stain were obtained from ThermoFisher Inc. and used as per the manufacturer's specification. TMRM (Tetramethylrhodamine, methyl ester) is a cell-permeant, cationic, red-orange fluorescent dye that is readily sequestered by active mitochondria and has been used for estimation of mitochondrial potential was used as described before (Sukumar, M., et al., Cell Metabolism, 23: 63-76 (2016)). Mitochondrial-ROS (mROS) was estimated by DCFDA (2′,7′-dichlorofluorescin diacetate) obtained from ThermoFisher Inc. Glucose uptake assay was done using 2-NBDG (2-(N-(7-Nitrobenz-2-oxa-1,3-diazol-4-yl)Amino)-2-Deoxyglucose). MitoFM Green was used for mitochondrial estimation by flow cytometery. All reagents were used as per the manufacturers' instructions.

Statistical analysis. Sample sizes were determined by prior experience and to achieve a confidence level of at least 95%. Statistical analysis was done using Microsoft Excel and GraphPad Prism6.0 as appropriate. Data were analyzed using two-tailed Student's t-test and two-way ANOVA as appropriate, and P<0.05 was considered significant.

### Results

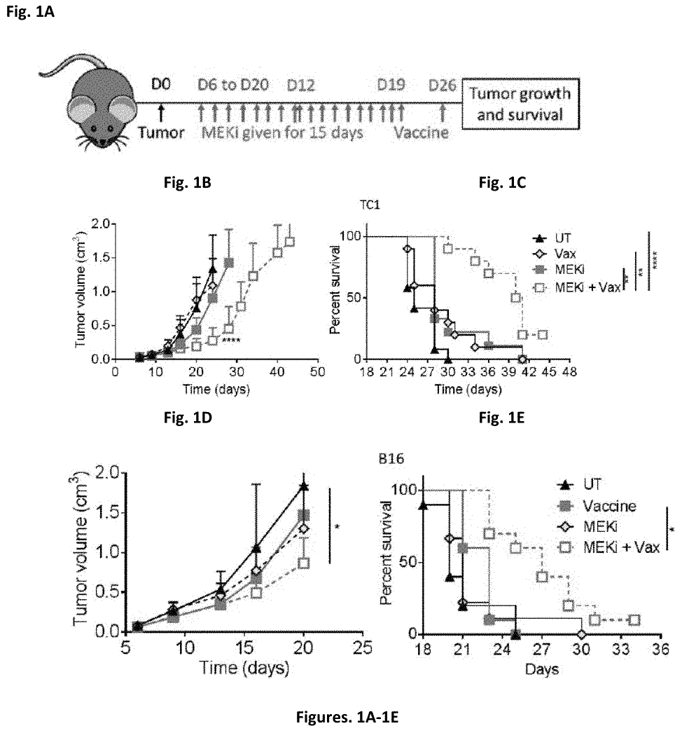

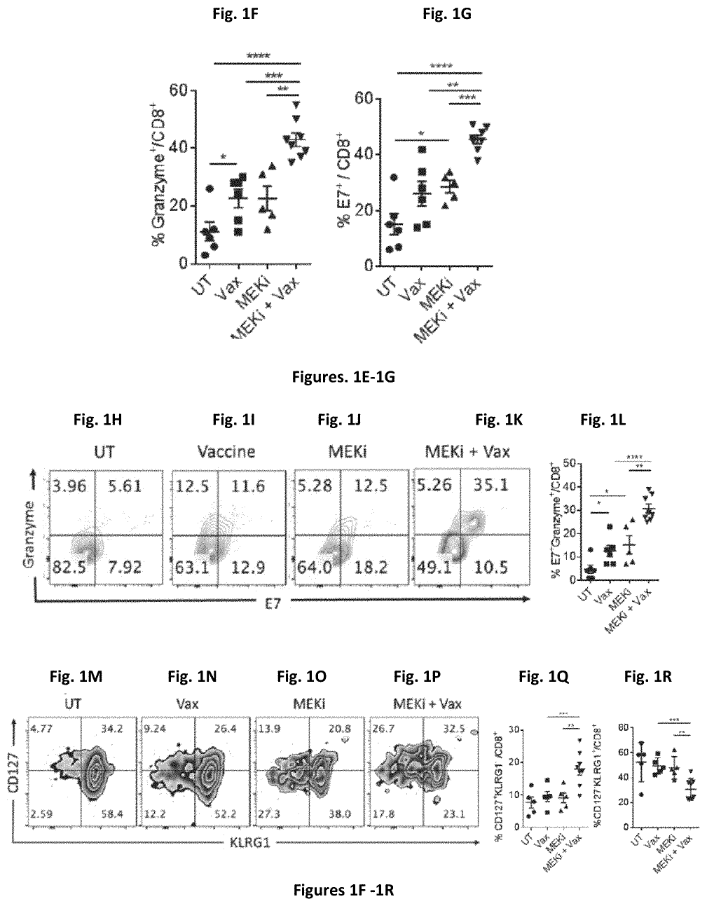

The immune effects of MEKi using Selumetinib, a MEK1/2 inhibitor (Troiani, T., et al., British Journal of Cancer, 106: 1648-1659 (2012)), were tested in two mouse transplantable tumor models, TC1 and B16 (FIG. 1A). Both of these tumor models carry specific antigens, HPV16 E7 and gp100 for TC1 and B16, respectively. Accordingly, vaccination with the specific antigen generates a measurable effector immune response, which helps in dissecting the effect of MEKi on the specific anti-tumor immunity. The data show that MEK inhibition significantly enhanced vaccine-specific effects in both the tumor models, leading to reduced tumor growth and increased survival (FIGS. 1B-1E).

To understand the mechanism by which MEKi enhanced the immune response, the effect of MEKi on the frequency of CD8⁺ T-cells in the TME was assessed. TC1 tumor-bearing mice treated with MEKi had a significant increase in the frequency of granzyme-secretion (FIG. 1F) and antigen-specific CD8⁺ T-cells generated by the E7-vaccine (FIG. 1G). Importantly, it was also observed a significant increase in the numbers of granzyme-secreting antigen-specific CD8⁺ T-cells (FIG. 1H-1L). This indicated that MEKi supported the expansion of functional effector cells in the TME generated by the vaccine. Since tumor-infiltrating lymphocytes are in a state of terminal differentiation and functional exhaustion and since it was found that MEKi enhances functionality, the effect of MEK inhibition on the phenotype of CD8⁺ T-cells in the TME was investigated. Compared to E7 vaccine or MEKi-alone, it was found that combination of MEKi and E7-vaccine led to a significant enrichment of memory-precursor CD127⁺ KLRG⁻0 (killer cell lectin-like receptor subfamily G, member 1) CD8⁺ T-cells (FIG. 1M-1Q). Moreover, MEKi+E7-vaccine treatment also resulted in reduced frequency of CD127⁻KLRG⁺ CD8⁺ T-cells (FIG. 1R), indicating that MEKi prevented the exhaustion of vaccine-induced CD8⁺ T-cells in the TME. Taken together, these results indicate that MEKi-treatment results in expansion of vaccine-induced anti-tumor antigen-specific effector cells. These results also highlight that MEKi decreases exhaustion and induces memory in CD8⁺ T-cells in the TME.

### Example 2: MEKi Treatment Induces Stem-Cell Memory CD8⁺ T Cells (T_{SCM})

### Results

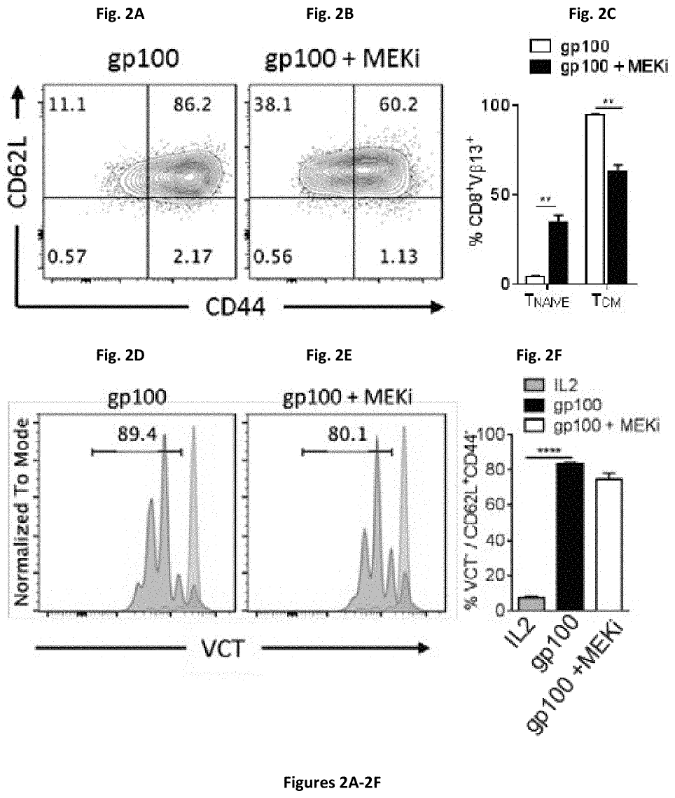

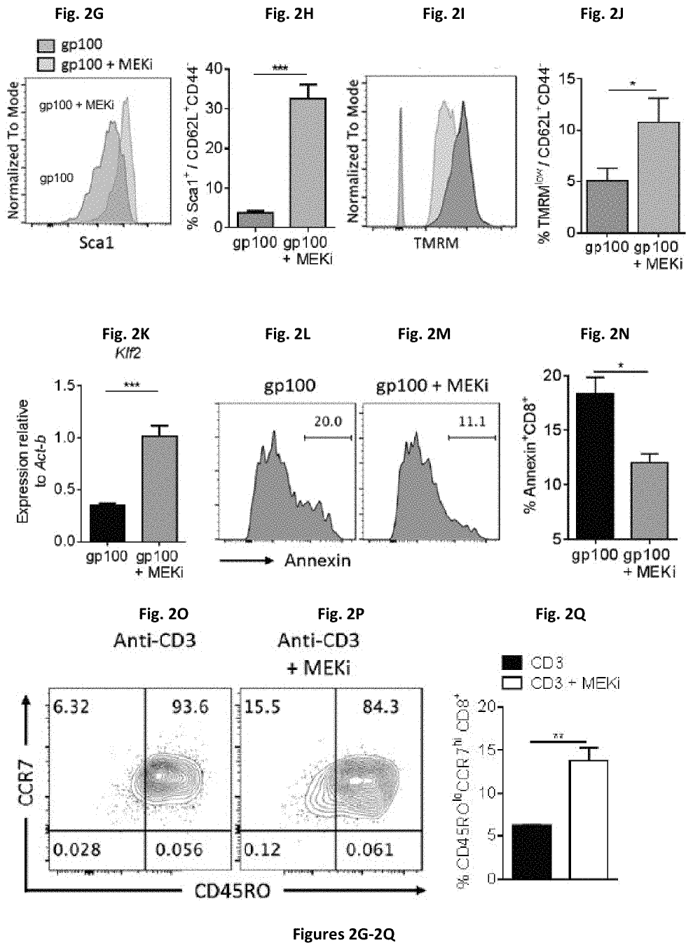

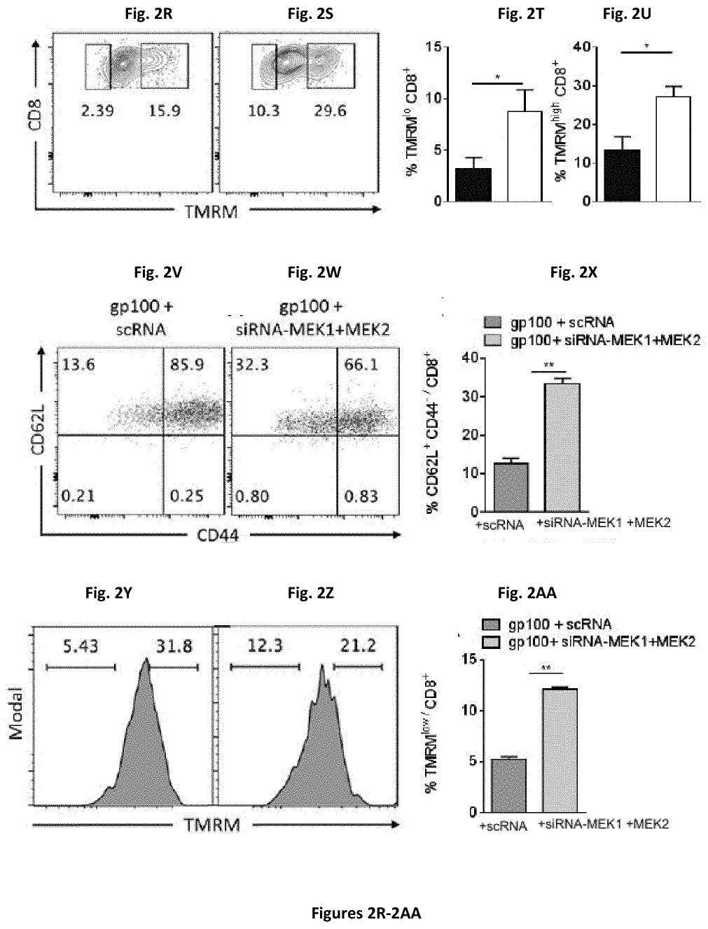

The in vivo data shown above clearly suggest that MEKi leads to an increase in memory CD8⁺ T-cells (CD127⁺ CD8⁺ T-cells) when effector cells are generated in antigen-treated animals (FIG. 1M-1P). To explore the mechanism by which MEKi influences the phenotype of CD8⁺ T-cells, CD8⁺ T-cells obtained from pMel-1 mice were activated with cognate gp100₂₅₋₃₃ peptide in the presence or absence of MEKi in vitro. As expected, it was found that, in the absence of MEKi, a major fraction of gp100₂₅₋₃₃ activated CD8⁺ T-cells attained a CD62L^{hi}CD44^{hi} central memory phenotype (T_{CM}) with only a minor fraction of the cells staying in the CD62L^{hi}CD44^{lo} na\ddot{ï}ve phenotype (FIG. 2A-2C). Surprisingly though, T-cell activation in the presence of MEKi resulted in a significant increase in the numbers of CD8⁺ T-cells with naïve-like phenotype (CD62L^{hi}CD44^{lo} (FIG. 2A-2C). However, unlike naïve CD8⁺ T-cells, CD62L^{hi}CD44^{lo}) cells generated after MEKi treatment showed robust proliferative ability (FIG. 2D-2F). These data show that the naïve-like CD8⁺ T-cells that were generated after MEKi treatment were physiologically active, indicating a distinct phenotype compared to naïve CD8⁺ T-cells generated in the absence of MEKi.

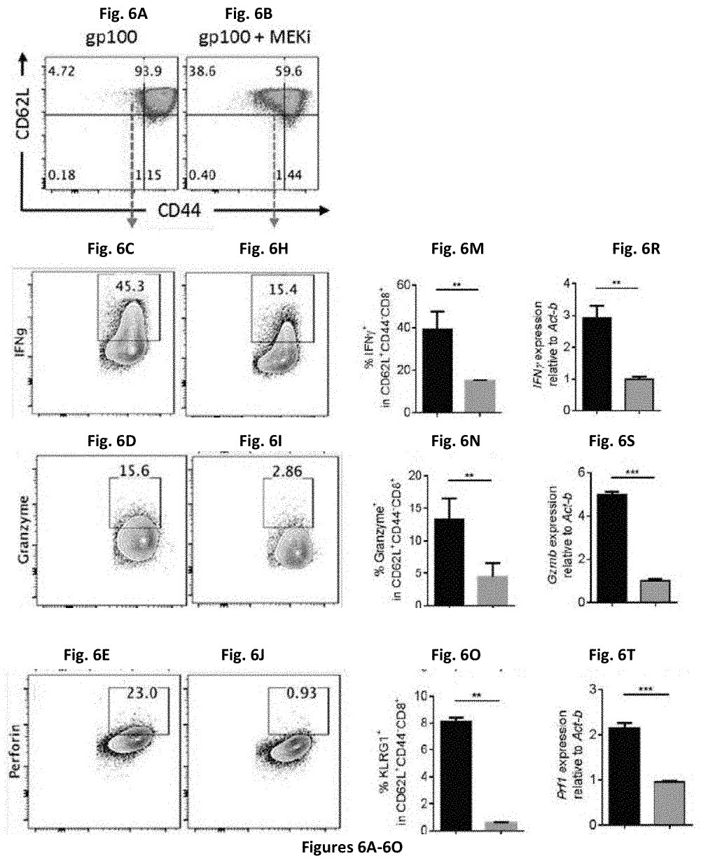

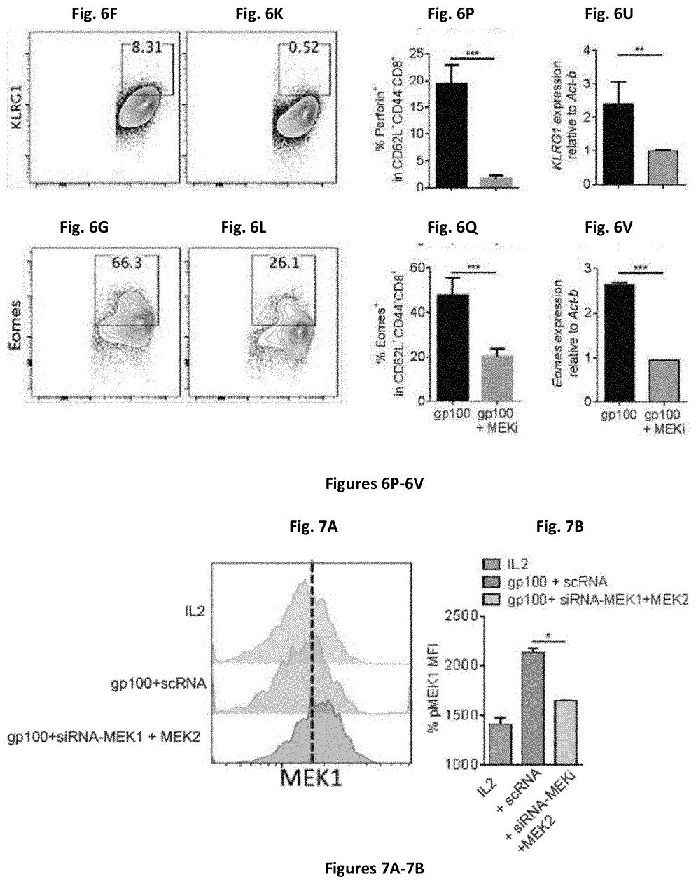

A minimally differentiated population of stem-cell memory (T_{SCM}) cells that produces a stronger effector cell population after antigenic re-challenge has been recently described (Gattinoni, et al., Nature Medicine, 17:1 290-1297 (2011)). In mice, these CD8⁺ T_{SCM} cells are characterized by a naïve-like phenotype (CD62L^{hi}CD44^{lo}) with high expression of Sca1 (Rosenblum, M. D., et al., Nat Rev Immuno, 16: 90-101 (2016)) and reduced mitochondrial potential (Sukumar, M., et al., Cell Metabolism, 23: 63-76 (2016)). Accordingly, it was found that the CD62L^{hi}CD44^{lo} naïve-like CD8⁺ T-cells that were generated after MEKi treatment expressed significantly elevated levels of Scal compared to untreated naïve cells (FIG. 2G-2H), and lower mitochondrial membrane potential represented by the higher frequency of TMRM^{low} T cells (FIG. 2I-2J). Furthermore, these CD62L^{hi}CD44^{lo} cells also demonstrated lower expression of markers associated with effector functions, including, IFNγ, granzyme and perforin, and with exhaustion, i.e., KLRG1 and Eomes, at both protein and mRNA levels (FIGS. 6A-6U). It is also known that expression of Kruppel-like factor 2 (Klf2) is associated with increased ability of T-cells to undergo self-renewal and prolonged survival by preventing apoptosis (Carlson, C. M., et al., Nature, 442: 299-302 (2006)), properties that are associated with the T_{SCM} phenotype. It was found that MEKi-treated CD8⁺ T-cells had higher expression of Klf2 (FIG. 2K) and a low tendency to undergo apoptosis (FIG. 2L-2N). Altogether these findings clearly indicate that activation of CD8⁺ T-cells in the presence of MEKi leads to induction of a distinct T-cell phenotype that is consistent with T_{SCM} cells.

The effect of the MEK inhibition on human CD8⁺ T-cells was tested. It was found that in agreement with the mouse data, MEK inhibition in activated human CD8⁺ T-cells led to a significant increase in the numbers of CD45RO^{lo} CCR7^{hi} CD8⁺ lymphocytes (FIGS. 20-2Q) and cells with low-mitochondrial potential (FIGS. 2R-2U). Both of these findings indicate a T_{SCM} cell phenotype in human CD8⁺ T-cells. These data strongly suggest that inhibition of the MEK1/2 during T-cell activation maintains T_{SCM} characteristics in a subset of CD8⁺ T-cells of mouse and human origin.

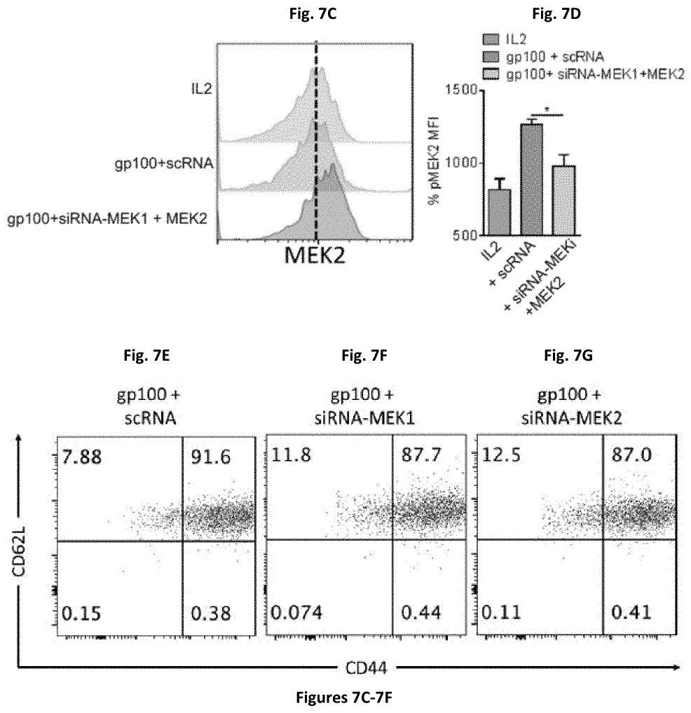

To further confirm the findings, genetic experiments were conducted by knocking down MEK1, MEK2, or both genes in pMel-1 CD8⁺ T-cells using specific siRNAs (FIGS. 7A-7D). Similar to the effect of pharmacologic MEK1/2 inhibition, the activation of MEK1/2 knock-down (KD) CD8⁺ T-cells led to a significant enrichment of naïve-like CD62L⁺CD44⁻ CD8⁺ T-cells (FIGS. 2V-2X) and a significant increase in the numbers of cells with lower mitochondrial potential (FIGS. 2Y-2AA). Interestingly, the KD of either MEK1 or MEK2 was not sufficient to produce the effect (FIGS. 7E-7G). These data further confirm that the inhibition of MEK1/2 signaling during activation of CD8⁺ T-cells led to enrichment of the T_{SCM} phenotype.

### Example 3: CD8⁺T_{SCM}-Cells Generated With MEK Inhibition Exhibit High Level of Metabolic Fitness

### Materials and Methods

Metabolic assays. For estimation of metabolic requirements, CD8⁺ T-cells were activated with gp100₂₅₋₃₃ peptide with/without MEKi for 48 hours followed by energy phenotype and mitochondrial stress tests (SeaHorse Bioscience) done as per the manufacturer's specifications. OCR and ECAR were measured with an XFp flux analyzer (Seahorse Bioscience). For all assays, 160,000 cell/ml were attached onto culture plates using Cell-Tak (BD Biosciences). OCR and ECAR were measure in unbuffered DMEM (Agilent Biotechnologies) supplemented with 10 mM D-glucose (Sigma-Aldrich), 10 mM L-glutamine and 2.5 mM pyruvate, as indicated. For certain experiments, after 48 hours of activation in the respective groups, cells were further exposed to anti-OX40-Ab for an additional 72 hours following OCR and ECAR estimation as described above. In a few experiments, cells were activated in the presence of oligomycin to block mitochondrial respiration or etomoxir, an inhibitor of fatty acid oxidation, followed by estimation of cell proliferation as an indicator of cellular activation.

### Results

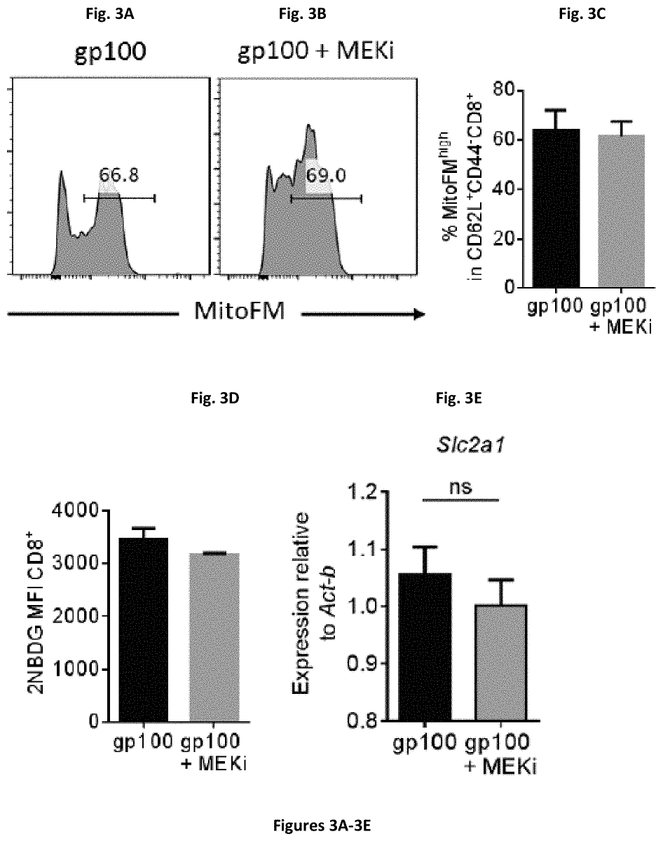

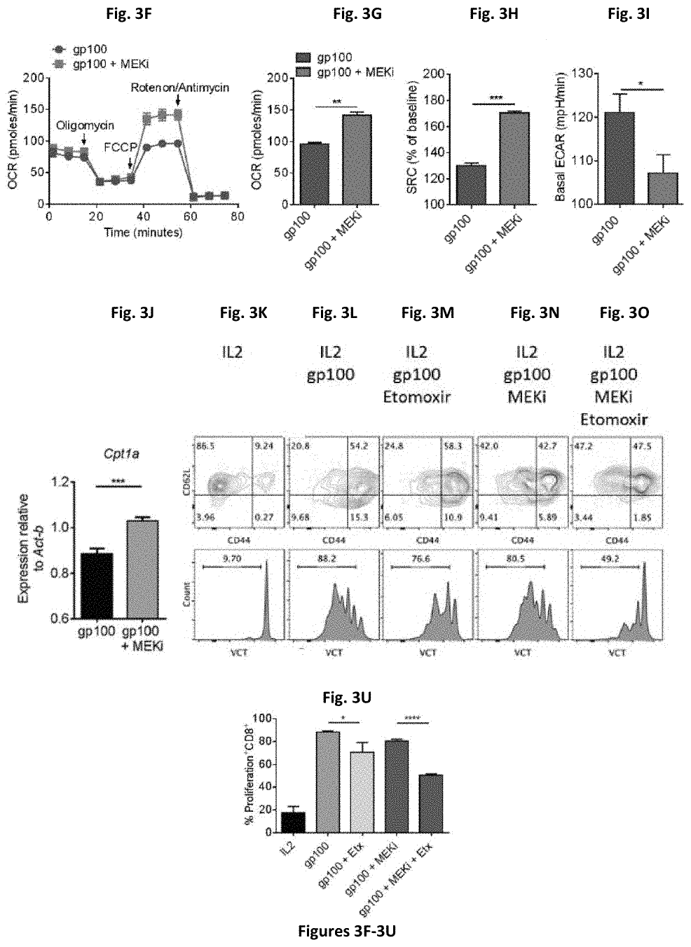

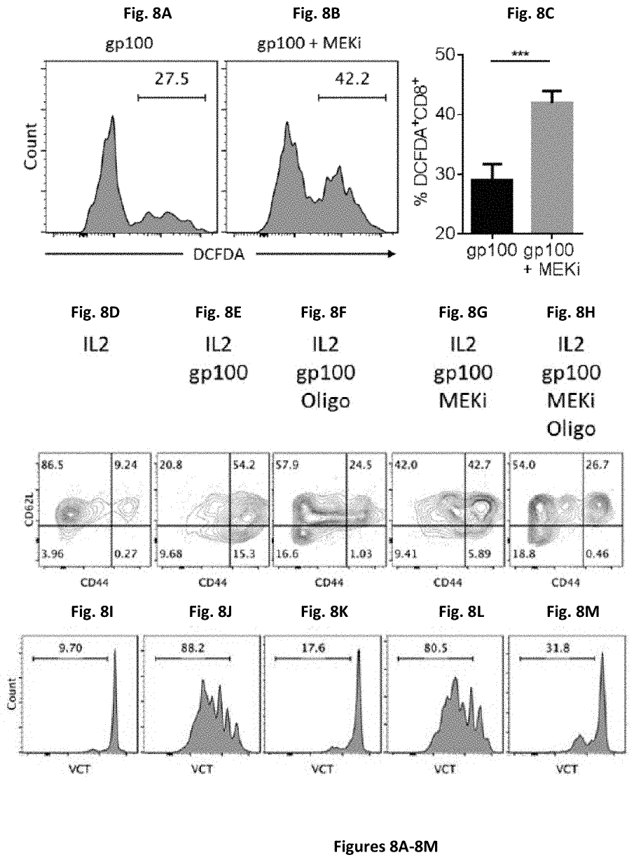

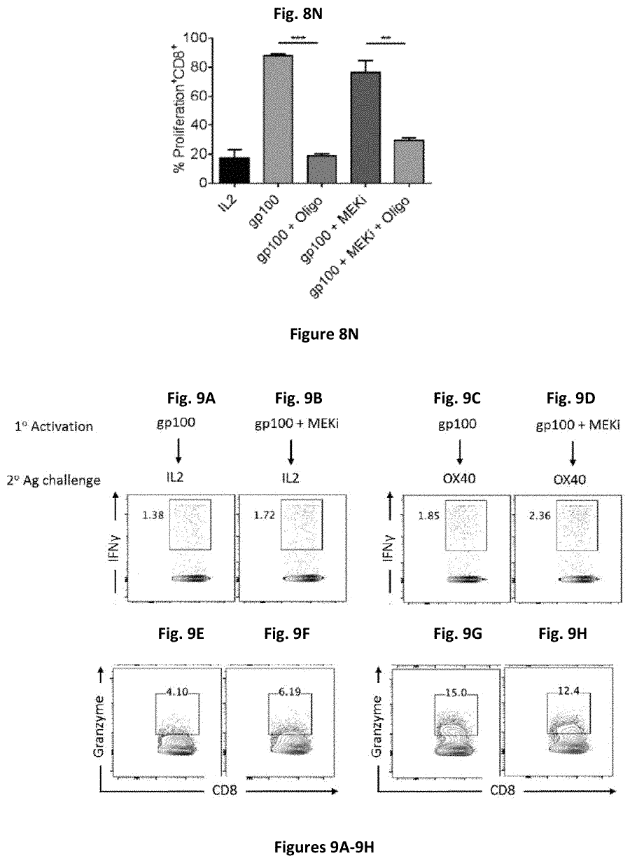

Metabolic fitness characteristics, including mitochondrial mass and function, glucose uptake and glucose utilization are key factors for anti-tumor activity of T-cells. Memory cells show a tendency to utilize OXPHOS and have an increased oxygen consumption rate (OCR), spare respiratory capacity (SRC), and mitochondria-associated reactive oxygen species (mROS) production. On the other hand, effector T-cells rely on cytoplasmic-aerobic glycolysis resulting in increased extracellular acidification rates (ECAR). However, the metabolic characteristics of T_{SCM} cells and the mechanisms regulating them remain unknown. Here, similar mitochondrial mass (FIGS. 3A-3C) and glucose uptake rates (FIG. 3D) were found in both MEKi-treated and untreated CD8⁺ T-cells. This was also confirmed by a similar expression of the glucose transporter gene Glutl (Slc2a1) (FIG. 3E). However, despite similar glucose uptake rates in the two cell populations, MEKi-treated CD8⁺ T-cells showed an increased mitochondrial respiration rate as shown by significantly higher OCR and SRC (FIGS. 3F-3H), and lower ECAR (FIG. 3I). This demonstrates a greater reliance of MEKi-treated CD8⁺ T-cells on mitochondrial respiration for energy production, indicating higher metabolic fitness. This was also confirmed by results showing an elevated production of mROS (FIGS. 8A-8C) and inhibition of proliferation in MEKi-treated CD8⁺ T-cells with oligomycin, a pharmacological inhibitor of mitochondrial-linked ATP synthesis (FIGS. 8D-8N).

Since metabolically fit cells are known to rely on fatty acid oxidation (FAO) as a mechanism for energy production, the reliance of MEKi-treated CD8⁺ T-cells on FAO was tested. An increased expression of carnitine palmitoyl transferase I (Cpt1), a rate-limiting enzyme required for FAO, was found in MEKi-treated CD8⁺ T-cells (FIG. 3J). This increased FAO in MEKi-treated pMel-1 CD8⁺ T-cells was also confirmed by a significant decrease in cell proliferation in the presence of etomoxir, a pharmacological inhibitor of FAO (FIGS. 3K-3U). Taken together, these results indicate a significant metabolic fitness in MEK-induced CD8⁺T_{SCM} T-cells.

### Example 4: CD8⁺ T-Cells Treated With MEKi Have Higher Recall Response Resulting in Superior Anti-Tumor Activity in Adoptive Cell Therapy

### Materials and Methods

Cell activation, drug treatment and adoptive transfer. FACS-sorted CD8⁺ cells from pMel-1 mice were activated with gp100 peptide (KVPRNQDWL (SEQ ID NO:2); 1 μM/10⁶ cells/mL) either alone or in combination with MEKi (500-1000 nM) in T-cell medium supplemented with 30 units of IL2 for 48 hours unless otherwise stated. In some experiments, at 48 h cells were washed and re-incubated in IL2-containing medium supplemented with gp100 and/or anti-OX40-Ab for 72 hours. For ACT experiments, CD8⁺ cells from pMel-1 mice were activated with gp100-peptide with and without MEKi for 48 hrs followed by transfer into 9-day-old B16 melanoma-bearing mice (3×10⁵ cells/mouse) that were treated with cytoxan (2 mg/mouse) at day 8.

### Results

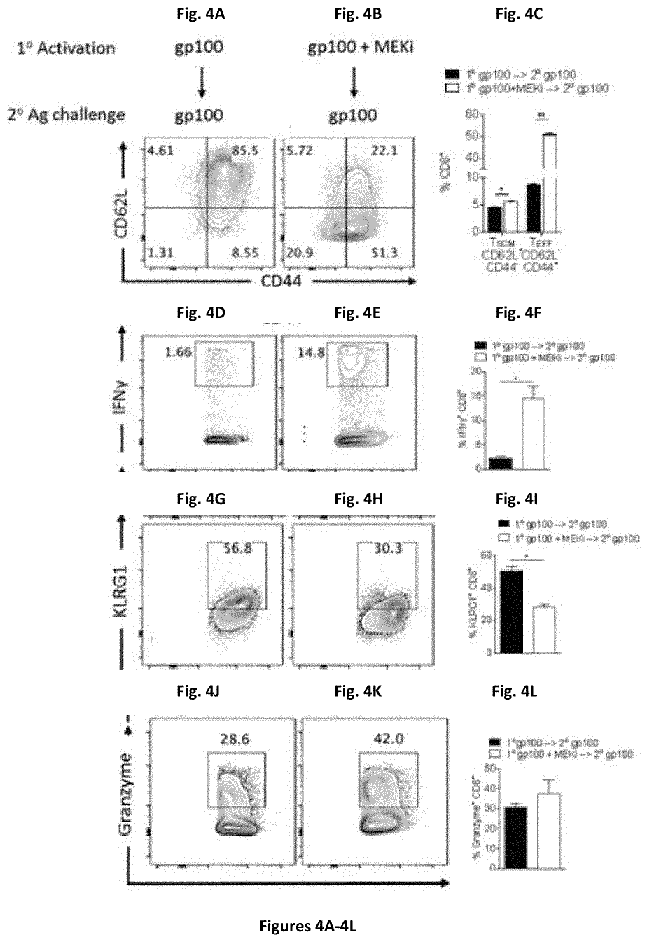

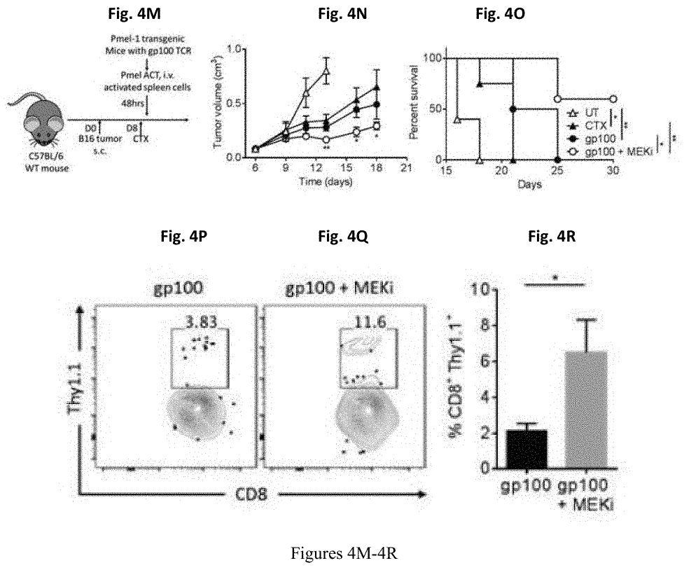

T-cells with high FAO and SRC are known to have stronger recall responses that are required for their effective anti-tumor activity, especially after adoptive cell therapy (ACT). Since an increased FAO and SRC was observed in MEKi-treated CD8⁺ T-cells, whether these cells will have higher recall response and, hence, superior anti-tumor activity when used in ACT in tumor-bearing mice was investigated. It was found that MEKi-treated CD8⁺ T-cells had more than 2.5-fold higher recall response measured in terms of expansion of terminal effector cells (CD62L⁻CD44⁺) (FIGS. 4A-4C). These granzyme B-expressing cells also expressed higher levels of IFNγ, and lower levels of KLRG1 (FIGS. 4D-4L). Thus, these results show that MEKi-treated CD8⁺ T-cells are highly activated with stronger recall response and less exhausted phenotype, indicating potentially stronger anti-tumor effects when used in ACT. To assess the anti-tumor effects of MEKi-treated CD8⁺ T-cells, gp100₂₅₋₃₃-activated pMel-1 CD8⁺ T-cells that were either treated or untreated with MEKi, were adoptively transferred in vitro into B16 tumor-bearing mice and evaluated the tumor growth rates and survival (FIG. 4M). It was found that, in contrast to mice that received untreated gp100₂₅₋₃₃-activated CD8⁺ T-cells, mice receiving MEKi-treated gp100₂₅₋₃₃-activated CD8⁺ T-cells had significantly higher anti-tumor response, resulting in tumor growth inhibition (FIG. 4N) and prolonged survival (FIG. 40). It was also found that 30 days after cell transfer, in contrast to untreated cells, MEKi-treated CD8⁺ T-cells were present with higher frequencies in the TME (FIGS. 4P-4R). In conclusion, these results indicate that MEKi-treated CD8⁺ T-cells have superior anti-tumor activity with prolonged persistence in the TME in the ACT setting.

### Example 5: MEK Inhibition Enhances the Anti-Tumor Effect of Agonist Anti-OX40-Ab By Stabilizing Memory and Promoting Effector Functions in CD8⁺ T-Cells

### Results

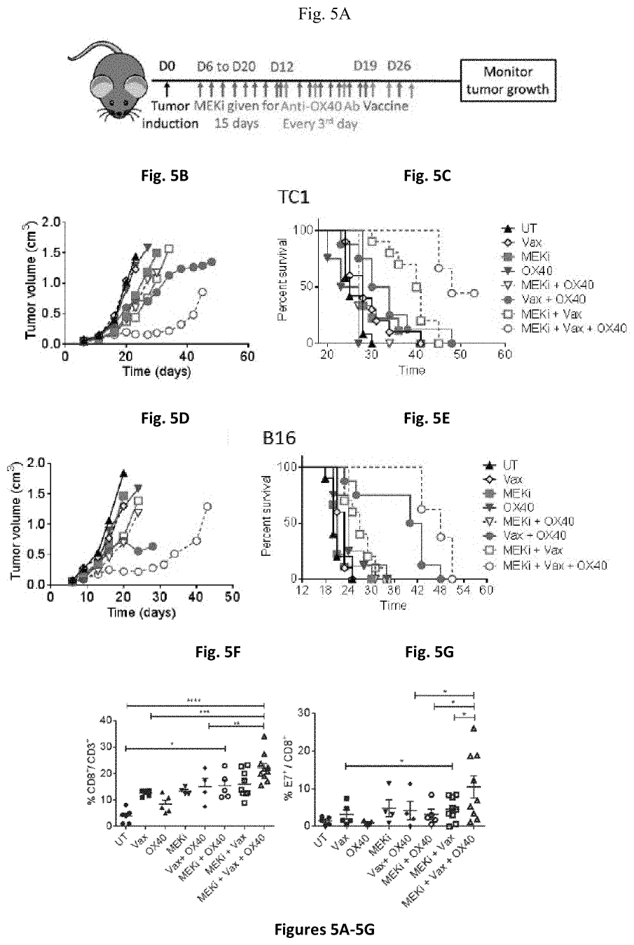

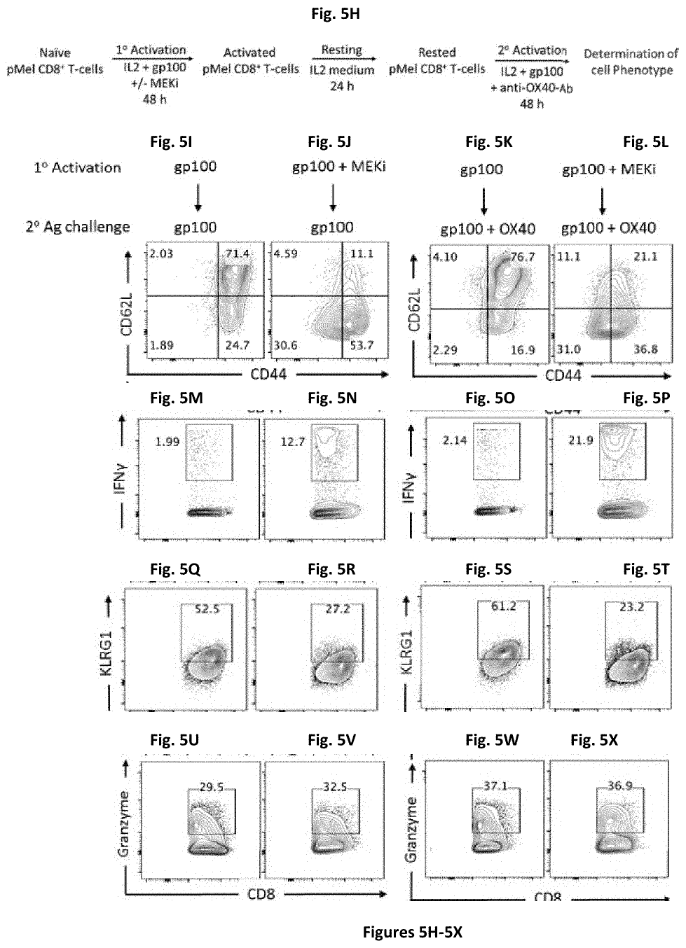

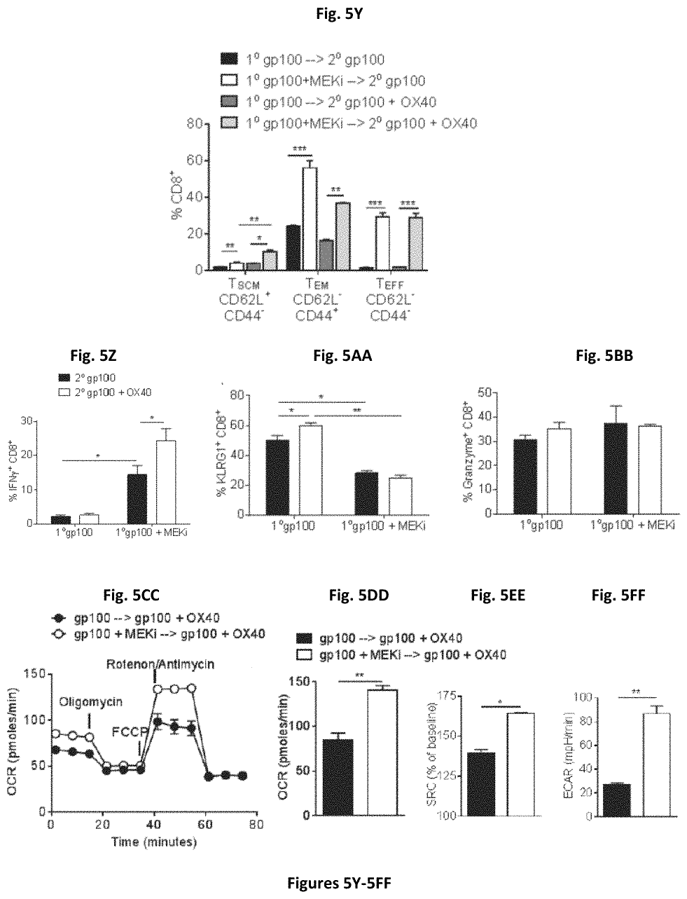

Activation of OX40 is known to stabilize memory and enhance effector functions in antigen primed T-cells (Sugamura, K., et al., Nat Rev Immunol, 4: 420-431 (2004)). Since MEKi induces T_{SCM} cells with higher recall response, it was believed that activation of OX40 would stabilize the T_{SCM} phenotype in MEKi-treated CD8⁺ T-cells and would exhibit a higher response to antigen stimulation. To test this hypothesis, TC1 and B16 tumor-bearing mice were treated with MEKi+vaccine (E7 or gp100₂₅₋₃₃ peptide, respectively) with or without anti-OX40 agonist Ab (FIG. 5A). It was found that even against large tumors (˜0.15 cm³) combination treatment using MEKi and anti-OX40-Ab resulted in a significant reduction in tumor growth and increase in mouse survival in both the tumor models. (FIGS. 5B-5E). Next, to define the mechanism of this synergistic anti-tumor response, the TME was examined for the infiltration of immune cells in the TC1 model. It was found that MEKi+E7-vaccine, in combination with anti-OX40-Ab resulted in a significantly higher infiltration of total (FIG. 5F) and of antigen-specific CD8⁺ T-cells (FIG. 5G) compared to when MEKi or anti-OX40-Ab were administered separately with the vaccine. These results highlighted the ability of MEKi to enhance the effect of anti-OX40-Ab and produce synergistic anti-tumor effects.

To further understand the mechanism by which MEKi enhances the effect of anti-OX40-Ab, the in vitro pMel-1 system was utilized to test recall capability of CD8⁺ T-cells. For this, pMel-1 CD8⁺ T-cells were activated in vitro with gp100₂₅₋₃₃ peptide in the presence or absence of MEKi, followed by overnight resting and then re-challenge with cognate antigen in conjugation with anti-OX40-Ab (FIG. 5H). It was found that MEKi-treated CD8⁺ T-cells generated significantly higher numbers of CD62L⁺ CD44⁻ cells (T_{SCM}) when treated with anti-OX40-Ab (FIGS. 5I-5BB). This indicated that anti-OX40-Ab was indeed preserving the T_{SCM} phenotype in CD8⁺ T-cells generated by MEKi. Moreover, anti-OX40-Ab treatment of MEKi-treated CD8⁺ T-cells also resulted in a significant expansion of CD62L⁻CD44⁺ T_{EFF} cells (FIGS. 5I-5L) that were producing high amounts of IFNγ and had less expression of KLRG1 (FIGS. 5M-5BB). Interestingly, the two cell populations (MEKi-treated and untreated) produced comparable levels of granzyme B upon rechallenge (FIGS. 5M-5BB). These data clearly suggest that anti-OX40-Ab treatment led to stabilization of the memory phenotype as well as induction of effector functions in MEKi-treated CD8⁺ T-cells. However, it is noteworthy that for induction of effector cells under the present experimental conditions, antigenic re-challenge was critical as none of the cell types (gp100₂₅₋₃₃ vs gp100₂₅₋₃₃+MEKi activated) produced IFNγ or granzyme B under basal conditions (FIGS. 9A-9D) as well as after anti-OX40-Ab treatment (FIGS. 9E-9H). T-cells with differential levels of activation may have distinct modes of energy production. Exhausted effector cells exclusively rely on aerobic glycolysis while non-exhausted effector cells utilize both OXPHOS and glycolysis for ATP generation. Since anti-OX40-Ab treatment of MEKi-treated CD8⁺ T-cells resulted in expansion of non-exhausted effector cells after antigenic re-challenge (FIGS. 5I-5L), the mode of energy production in these cells was investigated. For this, pre-activated CD8⁺ T-cells in respective groups (gp100₂₅₋₃₃ vs gp100₂₅₋₃₃+MEKi) were re-challenged with gp100₂₅₋₃₃+anti-OX40-Ab followed by estimation of energy phenotype (FIG. 5H). It was observed that MEKi-treated CD8⁺ T-cells, after gp100+anti-OX40-Ab treatment, resulted in increased OCR (FIGS. 5CC-5DD) and ECAR (FIG. 5FF) levels, indicating that these cells indeed were utilizing OXPHOS as well as aerobic glycolysis for energy generation. Moreover, it is important that anti-OX40-Ab treatment of MEKi-treated CD8⁺ T-cells also resulted in enhanced SRC (FIG. 5EE) that helps in induction of effector functions and maintenance of memory in T-cells. Thus, these results indicate that anti-OX40-Ab treatment of MEKi-treated CD8⁺ T-cells supported the expansion of non-exhausted effector cells that resulted in enhanced anti-tumor activity seen after combinational treatment.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method for reducing tumor burden in a subject in need thereof, comprising:
contacting CD8⁺ T-cells ex vivo with an effective amount of a MEK1/2 inhibitor to induce a CD62L^{hi}CD44^{lo} naïve-like phenotype in the CD8⁺ T-cells;
optionally expanding the induced CD8⁺ T-cells in culture; and
administering the induced CD8⁺ T-cells to the subject in an amount effective to reduce tumor burden in the subject.

2. The method of claim 1, further comprising administering to the subject an effective amount of an immunostimulatory agent, a potentiating agent, or a combination thereof.

3. The method of claim 2, wherein the CD8⁺ T-cells are genetically engineered CD8⁺ T-cells.

4. The method of claim 3, wherein the CD8⁺ T-cells are genetically engineered to express chimeric antigen receptors.

5. A method of adoptive cell transfer comprising
contacting CD8⁺ T-cells ex vivo with an effective amount of a MEK1/2 inhibitor and an immunotherapeutic agent to induce a CD62L^{hi}CD44^{lo} naïve-like phenotype in the CD8⁺ T-cells;
optionally expanding the induced CD8⁺ T-cells in culture; and
administering the induced CD8⁺ T-cells to the subject in an amount effective to reduce tumor burden in the subject.

6. The method of any one of claims 5, wherein the CD8⁺ T-cells are autologous CD8⁺ T-cells.

7. The method of claim 6, wherein the CD8⁺ T-cells are heterologous CD8⁺ T-cells.
